(19) United States
(12) Patent Application Publication
(10) Pub. No.: US 2003/0234022 A1
(43) Pub. Date: Dec. 25, 2003

(54) Single plate mandibular protrusive orthotic and method of manufacturing same

(76) Inventor: William A. Belfer, Ocean, NJ (US)

Correspondence Address: ROBERT M. SKOLNIK, 353 Monmouth Road, PO Box 22, West Long Branch, NJ, 07764-0022, US

(21) Appl. No.: 10/176,448

(22) Filed: Jun. 20, 2002

(51) Int. Cl. A61F 5/56

(52) U.S. Cl. 128/861

(57) ABSTRACT

A component assembly for a mandibular protrusive plate orthotic has an extraoral adjustable maxillary lip shield component and a method of joining the assembly to moldable and shapeable, self-curing or heat cured thermoplastic or thermoset materials, light cured composites, and to heat softened thermolabile elastomeric materials. Another embodiment of the assembly uses a maxillary dental plate as a resistance against the upper anterior maxilla The component assembly makes it possible for a protrusive lower plate to become a mandible and tongue advancement device and still maintain the inherent properties of a true dental orthotic.

### BACKGROUND OF THE INVENTION

1. Field of the Invention

The present invention relates to a single plate mandibular protrusive orthotic and a method of manufacturing same.

2. Description of the Related Art

Protrusive orthotics are used in dentistry to reposition the lower jaw into a more forward relationship with the upper jaw. A prerequisite of a protrusive orthotic plate is that (1) it must enable either one of the opposing dental arches to contact the biting surface of the orthotic, and (2) the protrusive plate must not be attached or interconnected with a resistance element in the maxilla. The biting surface of the protrusive orthotic may be made of hard or soft thermoplastic material, laminated of both hard and soft layers, and it can be made to various thicknesses. The purpose of a protrusive plate orthotics is to redirect the closing position of the lower jaw and distribute biting pressures uniformly about the dental arch, and at the same time hold the mandible in a forward position. They may also be used to increase the oropharyngeal airway by repositioning the mandible and tongue anterirorly for the treatment of obstructive sleep apnea and snoring. They are also used to treat muscle and joint pain and dysfunction associated with temporomandibular joint disorder, bruxism, and mal-alignment of the jaws.

The prior art describes protrusive orthotic devices that are retained by fitting to either one or both jaws. Several of them are capable of holding the protrusive jaw position in either the upright or supine position. However, there are no dual plate devices that can hold the jaw in protrusive position connected by a hinge, a post, a hook or flexible straps to dental plates that are retained upon the opposing jaw. There are also no single plate orthotics that allow complete freedom of mandibular motion in the protrusive position during sleep in the supine or lateral position. Furthermore, protrusive orthotics that rely on some type of interconnection between opposing plates limit the ability of the lower jaw to move normally and unfettered. The constraint of normal jaw motion and the influence upon the temporomandibular joint (TMJ) by mechanical devices can be problematic for TMJ health. Ideally, a protrusive orthotic should not dictate any tempormandibular joint position other than the normal protrusive path of the mandible.

Frantz, et al., U.S. Pat. No. 6,109,265, describes a double plate orthotic that has interchangeable elastic connectors which hold the relative posture of the lower jaw. They utilize vacuum formed plastic sheets and form them into retentive plates upon models of the patient's upper and lower teeth. This procedure is performed with a thermo-pressure machine. Thereafter, the method establishes the protrusive position of the mandible by the addition of inclined wedges that are affixed to the biting surface of the plates with cold curing acrylic bonding material. The mechanism that holds the mandible protrusively in the inclined position is a pair of interarch elastic straps which are affixed to the sides of the plates.

Franz, et al. U.S. Pat. No. 5,7964,627, also describe a positioning device that consists of upper and lower interconnected impression trays that are attached to an adjustable strap, which extends outside the mouth. The strap enables the lower tray to be advanced and then stabilized into the upper tray so that it can aid in determining the forward position of the mandible. This device is helpful in making a permanent orthotic in the laboratory, but by itself it is not an orthotic.

Lowe U.S. Pat. No. 5,409,017, Halstrom U.S. Pat. No. 5,365,945 and Parker U.S. Pat. No. 5,267,862 describe double plate mandibular advancement devices that are joined by adjustment screws or jigs, but these devices have no extraoral components. Kidd U.S. Pat. No. 5,829,441 describes a device of upper and lower plates that are custom fitted to the patient by heating and directly molding to the patients teeth; the protrusive adjustment is made via altering the length of a piston/tube assembly, which is fixed to the lateral sides of the customized intraoral plates.

There are other devices as in Hayes, et al. U.S. Pat. No. 5,092,346 and Meade U.S. Pat. No. 5,277,202 that are single plate protrusive devices, that are unhinged, and which have no extraoral components. These devices are manufactured in a generic form with thermolabile elastomeric material that is either chemically bonded or fused to a hard skeleton of carboxylate, polyvinyl or acrylic, as in Hayes et al. U.S. Pat. No. 5,092,346, or they are made entirely of a soft durometer, thermolabile elastomer, as in Meade U.S. Pat. No. 5,277,202. Both of these retain the mandible in protrusive position by way of a ramp that is built into the undersurface of the upper plate. The ramp of thermolabile elastomer is molded to the back surface of the lower anterior teeth, and thus the orthotic resists rearward relapse of the mandible after the mandible is positioned protrusively into it.

The Thornton appliance (Thornton U.S. Pat. No. 5,755,219) consists of upper and lower hinged plates and it has an extraoral adjustment screw component. It also has a “J” shaped hook at the intraoral portion of the adjustment screw, which engages a transverse bar that is located in the lower plate behind the incisor teeth. With the Thornton's device, the resistance for holding the lower jaw forward is the upper dentition. There is no extraoral resistance component to this device. This appliance is manufactured as two independent plastic trays, which are filled with a layer of heat moldable elastomeric material. The moldable material is heated to a softened state and placed over the individual dental arches to form a detailed impression of the teeth—allowed to cool to a set—and removed from the mouth. In U.S. Pat. No. 6,305,376 Thornton describes a slotted plate that is engaged by an adjustable engager to forwardly and vertically position the mandible relative to the maxilla. The engager is integrated with an adjustable screw assembly, which is in turn molded into the upper plate.

Clark U.S. Pat. No. 5,871,350 describes an adjustable post and tube assembly which when attached to the lateral surfaces of an upper and lower plate or to a fixed attachment upon an individual tooth is capable of advancing the mandible. This too is an intraoral assembly.

Gravity, nocturnal clenching and tooth grinding (i.e. bruxism) work against the ability of any orthotic to hold the lower jaw protrusively while the head is in the supine position. Thus, many devices require that the lower jaw be pitted against a very retentive upper plate. Double plate devices, interpose a greater interarch (between the jaws) thickness which may adversely increase the vertical positioning of the jaws. They also prevent the natural dentition from articulating upon surface indentations of the opposing dental arch and special surface elevations and contours that may be helpful to influence mandibular function cannot be incorporated into the contacting plates. An important distinction of single plate mandibular orthotics is that they enable the clinician to change the vertical depth of the bite (distance of vertical closure) at any time after the device is fabricated. The biting surface can be altered by the addition of various inclines and depressions. Another important advantage of single mandibular plate orthotics is that they enable the device to be made by direct (no dental molds) methods. Thus, they are less costly and they save time.

Single plate protrusive orthotics may use either the upper or lower jaw as a point of fixation. However, there is a disadvantage to a maxillary plate orthotic which uses (1) the upper jaw as the area of fixation and (2) the upper plate as the origin of an inclined ramp to the lower jaw or plate. Even though such devices are able to protrude the lower jaw they do not allow the contact surfaces of the device to disarticulate the lower teeth during periods of nocturnal bruxism. Since they reduce occlusal pressure by disarticulation and complete freedom ofjaw motion, mandibular protrusive orthotics are more advantageous for reducing pressures upon the tempormandibular joint during nocturnal bruxism.

The ability to construct an orthotic rapidly and without the need for taking dental impressions is a desirable feature of an orthotic because it is a more efficient treatment and it is less costly. However, hitherto soft durometer thermolabile elastomer materials were the materials used for this process. The disadvantage of these so called “boil and bite orthotics” is that they are subject to distortion, the soft durometer material is not durable and the biting surface cannot be customized with indentations and inclines as hard thermoplastics can. In addition, they are injection molded generic devices, which may not be as retentive as custom laboratory fabrications. Protrusive orthotics, which are made of hard durometer materials, are, in the prior art, made only in the dental laboratory and impressions are required. It is therefore and advantage to have a means of making a protrusive orthotic that has the properties of either softness or hardness and can me assembled in the dental operatory in one visit by a direct impression method.

Sullivan, U.S. Pat. No. 4,519,386, and Norton, U.S. Pat. No. 4,671,766, both describe devices that are intended to limit jaw motion in order to achieve a relaxation and healing of the masticator muscles. In the Norton art, maxillary and mandibular dental plates cover the dentition. The plates are separated when the jaws are in a relaxed state, but they become engaged only during closing of the mouth via lateral flanges, which extend vertically from the sides of the respective plates. The interlocking of the flanges limits lateral motion of the jaw without limiting protrusive or vertical motion. The Sullivan device consists of a palatal plate which overlays only upon the upper molar teeth; the molar overlay section has lateral walls that extend inferiorly on the medial and lateral side of the upper molars area. The walls prohibit lateral motion, but not vertical motion when they engage the lateral surfaces of the mandibular molar teeth.

Boyd U.S. Pat. Nos. 5,513,656 and 5,085,584 describe a device and a method of adding self-curing thermoplastic material to a segmental tray of an adherent substrate, and a method of customizing it directly in the patient's mouth. The tray is made from an adherent substrate. The purpose of the technique is to custom form a jig, which he describes in U.S. Pat. Nos. 5,513,656 and 5,795,150 that will open the bite and advance the mandible. The Boyd device has no protrusive mechanism with which to facilitate protrusion of the mandible, however, the device will disarticulate (disclude) the teeth during episodes of bruxism or normal function.

Boyd teaches that the discluder device consists of a dome shaped element that is either situated upon the incisal most surface of the template upon the teeth, or mounted from a maxillary plate to contact with the incisal edge of one or more lower incisor teeth. The Boyd method discludes the lateral and posterior teeth in when the jaws are in closure or in functional activity, so that the posterior teeth do not touch and the bite remains open during the time of wear of the device. Boyd's discluding element has no ability in itself to protrude the lower jaw. However, the principle upon which it is based is that disclusion of all of the teeth, except the maxillary and mandibular incisor teeth during function and/or jaw closure shall reduce the nociceptive trigeminal reflex within the masticator muscles and thereby reduce muscle contracture and spasm in these muscles. Accordingly, it is an advantage to incorporate this principle into a protrusive device. The ability to reduce masticator muscle contracture at the same time as protruding the mandible has hitherto not been taught.

Belfer describes mandibular orthotic which has an extension member fixedly connected to the front of a dental overlay and wherein the extension member is a flexible strip of plastic, U.S. Pat. No. 5,720,302. The dental overlay covers all of the lower teeth and is made from elastomeric material, which is molded together with the extension member as a single unit. An extraoral lip shield is adjustable horizontally upon the extension member. The shield cannot be vertically adjusted upon the extension member as there is no vertical sliding bracket component associated with it.

Belfer U.S. Pat. No. 5,810,013 describes a protrusive anti-snoring orthotic with an external lip shield wherein there is an L shaped bracket, which slides horizontally upon an extension member that is fixed to a dental overlay on the mandibular teeth. The L shaped bracket has an opening or channel, which receives the vertical leg of the lip shield on one end and receives the horizontal extension member on the horizontal channel. The bracket is stabilized upon the extension member by means of a screw. However, the L shaped configuration of this channeled bracket necessitates that it be made from rigid materials, which are more difficult to adapt and bend. With a channeled bracket, changing the angulation is difficult.

Alternatively, the art of U.S. Pat. No. 5,810,013 shows a retentive band that holds an L shaped bracket. The bracket has a series of equally spaced ridges on its bottom aspect that engage a series of matching grooves that are situated on the upper surface of he extension member. As the holding band is slipped upon the horizontal leg of the L bracket and the extension member, it holds the horizontal positioning to the shield firmly in place via interlocking of the grooves and ridges that are designed onto the opposing surfaces of these parts. However, once it has been positioned as such, the lip shield cannot be conveniently repositioned. The protrusive posture of the mandible cannot be changed without removing the holding band from the assembly, repositioning the bracket further back toward the dental plate, and then replacing the holding band again once the desired amount of mandibular protrusion has been achieved.

In U.S. Pat. No. 5,921,241, Belfer shows that the vertical connecting member of he L shaped bracket is connected to the lip shield. The horizontal portion of the bracket is slidably received by a holding ring in a co-linear relationship. In addition, the horizontal portion of the bracket has a series of ridges on its undersurface, which engage ridges upon a channel upon the superior surface of the extension member.

The art of Boyd is not applicable to the protrusive assembly of Belfer. A member that is mounted or fabricated upon the superior contact surface, above the plane of the dental overlay in the Belfer device, might cause vertical depression of the mandible sufficiently enough to reduce vertical height adjustment of the lip shield. For example, a 3 millimeter elevation of the incisor area above the superior surface of the extension member would lower the mandible upon bite closure three millimeters and result in a three millimeter reduction of the maximum height adjustment of the lip shield beneath the nose. More importantly, in order for the Belfer component assembly to maximize its protrusive function, the sliding bracket is mounted flush and in the same horizontal plane as the flat contact surface of the extended member so that it can slide freely in a rearward direction, thereby maximizing the potential to advance the mandible. Any addition to the thickness of the dental plate surface in the anterior segment behind the extended member of the Belfer device will limit the horizontal and rearward translation of the angled sliding bracket and lip shield Belfer teaches in this new art, that achieving disclusion of the posterior teeth with the protrusive component assemble that utilizes an external sliding lip shield can only be achieved reducing the posterior surface thickness of the mandibular dental overlay and maintaining the height of the dental overlay in the incisor contact area. Therefore, when the upper incisor strikes the anterior portion of the lower plate, the posterior teeth are kept in disclusion in all ranges of functional motion and in static protrusive closure. In this way the Belfer component assembly can render a protrusive dental plate capable of attaining the same functionality with regard to disclusion of the cuspid and molar teeth as are taught by Boyd (U.S. Pat. Nos. 5,513,656 and 5,085,584). The hard acrylic resin composition of the dental overlay makes it very suitable to grinding and lowering the surface heights of the plate in the posterior area by 3 millimeters, while allowing the original pre fabricated surface to remain at the higher level. In addition, the overlay can be pre-fabricated with the incisor contact area higher than the posterior area.

### SUMMARY OF THE INVENTION

The present invention is a component assembly that is utilized to manufacture a mandibular protrusive orthotic. The assembly can be either be hybrid into (1) a single mandibular plate with an extraoral adjustable lip shield component or (2) a dual plate mandibular protrusive orthotic, that replaces the external lip shield with an independent maxillary dental plate. The present invention also discloses a method of joining this component assembly to moldable and shapeable, self-curing or heat cured thermoplastic or thermoset materials, light cured composites, and to heat softened thermolabile elastomeric materials. In either the single plate or double plate form the component assembly makes it possible for a protrusive lower plate to become a mandible and tongue advancement device with out impairing normal mandibular function and without a structural connector to the upper jaw.

The design of the component assembly enables the active adjustment mechanism be molded into a dental plate over the lower dental arch in order to construct a single plate mandibular protrusive orthotic; in order to be joined to generic “one size fits all” trays; or trays of different sizes. The material properties of the assembly enable its biting surface to be customized to create specific contact relationships with the opposing jaw. Hitherto, no protrusive dental appliance was capable of combining mandibular advancement with disclusion of the cuspid, bicuspid and molar teeth during complete jaw closure and/or functional excursive motion.

This orthotic resists placing reciprocal pressures on the upper teeth. In the second embodiment, the external maxillary component is a soft tissue borne lip shield while the mandibular dental plate buttresses against the dense cortical bone and gingiva of the inner aspect of the lower jaw. In the second embodiment the maxillary dental plate unitizes upper teeth, and this makes a more stable resistance unit. The advantage is providing these alternative means of resistance, i.e. the lip shield vs. the maxillary dental plate give the clinician the option of choosing the most suitable anchorage for the patient's dental condition. Because there is no interconnection between the jaws in either embodiment, the benefits of a true dental orthotic, such as interarch adjustment of the biting surface, and alteration of vertical interdental thickness, can be realized in simple chairside techniques.

The prior art does not describe a protrusive orthotic which possess these characteristics; that also could be made in a single visit method.

There has also been no mandibular protrusive orthotics that could be made directly in the mouth from a hard or semi-soft durometer thermoplastic material.

Further objects and advantages of the invention are:

a. The lip shield is buttressed against the soft tissue and underlying cortical bone of the maxilla and thereby eliminates tooth-moving pressures from being applied to the upper teeth. Since there is no interconnection, the path insertion of either form of the device into the mouth is easily performed.

Alternatively, a maxillary dental plate can replace the lip shield component in order to provide an intraoral means of anchoring the mandible in protrusive position, in which case the lip shield is not necessary.

The maxillary dental plate has no active function other than to integrate the dental arch as a stable unit which can anchor the mandibular advancement. The maxillary dental plate can be laboratory made from vacuum formed polyvinyl materials or in the dental laboratory from resinous methacrylate.

b. This orthotic can be serially adjusted without removal from the mouth.

c. The mandibular overlay is chemically bondable to, acrylic, methyl vinyl acetate, carboxylate, polycarbonate, polypropylene, polyethylene, and polyvinyl, glass-filled composites

d. The mandibular overlay is mechanically bondable to copolycarolactone, caprolactone, natural and synthetic rubbers, ethylene vinyl acetate, methyl vinyl acetate, polyurethane, polyvinyl, vinyl, silicone.

e. The component assembly can be manufactured in simple chairside methods, which involve direct customization in the mouth. This important property enables the biting surface of the overlay to be customized, depending on treatment objectives, so that (1) the vertical separation of the jaws can be modified, (2) the path of jaw closure can be controlled, (3) and the excursive motion of the lower jaw can be altered to disengage (disclude) the posterior teeth.

f. The mandibular overlay may be either a single plane, flat biting surface or a two plane biting surface in its pre-customized form. In the latter form, that portion of the overlay which generally contacts only with the upper incisor teeth is thicker than the remaining posterior. In addition, the overlay may be customized by the dentist. Thus, it can have surface characteristics added to it to influence specific jaw motions and cause disarticulation of the teeth during bruxism and clenching of the teeth.

g. The overlay is a hard durometer resin that can be combined into the mandibular dental plate with hard or semi-hard or soft durometer materials.

h. The component assembly is easy to customize to various jaw discrepancies and extreme relationships without removing it from the mouth.

i. The component assembly enables the protrusive orthotic to give the mandible and the mandibular condyles full range of motion (while dual plates and some upper single plates do not.)

j. The component assembly enables the orthotic to be adjustable vertically as well as horizontally to suit various anatomical relationships and therapeutic objectives.

k. The vertical thickness of the plate can be increased to open the vertical separation of the jaws.

l. The component assembly has a simple locking mechanism that allows the operator to easily serially adjust the protrusion of the mandible.

m. The component assembly can be made at chair side with a material that can be formulated to various flexibilities and elastic properties.

n. The orthotic can be made of materials, which allow it to be easily removed from the mouth, and warmed to a softened state for easy insertion into the mouth.

o. The dental overlay has retentive properties by virtue of its chemical composition and physical design that enable it to be attached to a soft durometer, resilient elastomeric material in the molar areas of the orthotic. The dual durometer orthotic to adsorb pressures from the biting of molar teeth during sleep bruxism that can harm the TMJ and associated masticator musculature. The hard durometer portion of the dental plate enables the dental plate of the orthotic to be manually customized by the to disclude the posterior teeth during bruxism, thus enhancing its overall effectiveness as an anti-bruxism, anti-TMJ device.

The foregoing, as well as further objects and advantages of the invention will become apparent to those skilled in the art from a review of the following detailed description of my invention, reference being made to the accompanying drawings.

### BRIEF DESCRIPTION OF THE DRAWINGS

FIG. 1 is a perspective view of the preferred embodiment of the invention;

FIG. 2 is a sectional view of the preferred embodiment taken along the lines 2-2 of FIG. 1;

FIG. 3 is an exploded view of the component parts of the preferred embodiment of FIGS. 1-2;

FIG. 4 is an exploded view of another embodiment of the invention; and

FIG. 5 is a perspective view of a modified component of FIGS. 1 and 4.

### DETAILED DESCRIPTION OF THE PREFERRED EMBODIMENT

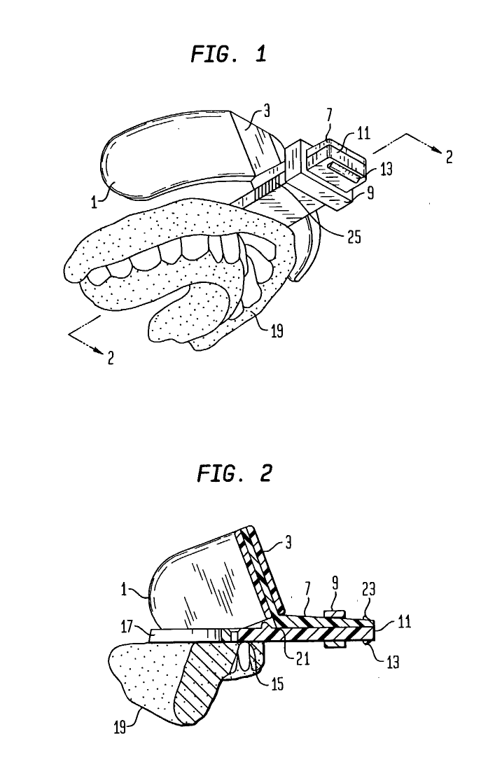

As shown in FIGS. 1-3, the preferred embodiment includes an adjustable concave maxillary lip shield 1. The shield has a flat vertical frontal portion 3 having a channel aperture 5 formed beneath the frontal portion 3. The channel aperture 5 receives the vertical portion of L-shaped bracket 7. The horizontal portion of the L-shaped bracket is attached to the extension member 11 with a holding band 9. A dental overlay 17 is attached to the extension member 11. A dental plate 19 is attached to the overlay 17 using holes 15.

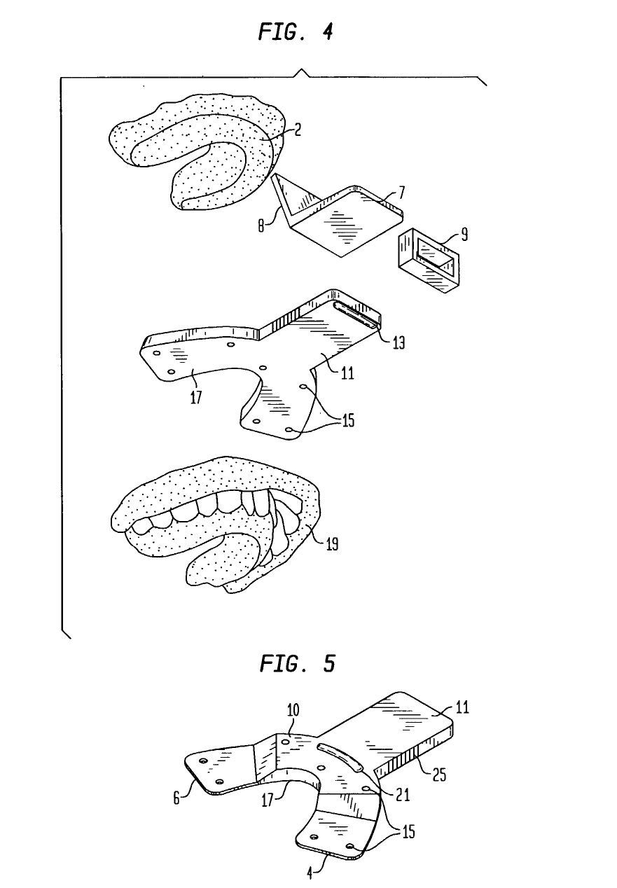

Two ridges 13 and 23 are formed on extension member 11 to prevent holding band 9 from sliding off the extension 11. A curved indicator line in the form of a ridge 21 (also shown in FIG. 4) is provided to aid in the positioning of the occlusal plate over the lower incisors. The line 21 on the superior surface of the occlusal plate 17 aids in the positioning of the occlusal plate 17 over the lower incisors. Serrations 25 are also formed in the sides of extension member 11 to further lock the holding band 9 in place as well as to provide indicia for noting the position of the lip shield 1 relative to the dental plate 19.

The L-shaped bracket 7 is slightly angled—15 degrees obtusely from the vertical plane—towards the lip. It is made from a flexible and bendable thermoplastic that has inherent memory and that can be adjusted to increase its angulation for better adaptability to the surface of the subnasal skin. This adjustment requires that the vertical leg of the bracket be warmed in hot water for 15 seconds, and then, holding the bracket in the desired angulation under cold water for a permanent set. By so doing the position of the concave lip shield may be fitted uniformly against the lip.

The concave lip shield 1 has a channel aperture 5 within the center of greatest width and this channel receives the vertical leg of the L-shaped bracket 7. The shield 1 is slidably adjustable vertically without requiring removal from the vertical bracket. This is an improvement over 5,810,13 because the added flexibility created by removing the channel design of the bracket imparts greater adjustability and fitting of the extraoral lip shield to the patient.

The horizontal leg of the L-shaped bracket 7 is slidably connected to the extension member 11 upon a smooth upper surface and, unlike the previous Belfer art, there are no ridges on the horizontal leg to engage the extension member. This enables horizontal adjustment of the lip shield and protrusive positioning of the mandible, by simply pushing the bracket rearward against the subnasal maxillary tissue while simultaneously protruding the lower jaw. Protrusion of the mandible is thus achievable without removing the orthotic from the mouth to reposition the parts or to remove the L-shaped bracket 7 from the extension member 2.

The vertical leg of the l-shaped bracket 7 can be trimmed with cutting shears so that one is able to reduce its vertical height in order to prevent the lip shield 1 from impinging beneath the nose; it can be fixed permanently in position by drilling a hole in the shield and vertical arm thru and thru and then filling it with a composite, hot glue EVA, or suitable acrylic compound. This was not possible with channeled bracket in the old art.

The horizontal leg of the L-shaped bracket 7 is tapered from the angle with the vertical leg to its terminal end. When the L-shaped bracket is 7 positioned upon the extension 11 and the holding band 9 is pushed rearward it binds on the horizontal wedge and thereby retains the sliding portion of the L-shaped bracket 7 in a fixed position. By loosening the holding ramp forward or backward adjustability of the bracket 7—lip shield 1 assembly is possible without removing the orthotic from the mouth.

The overlay 17 consists of a U-shaped portion that conforms to the average shape of the human dental arch and this part positions over the bite surface of the lower dentition. The extension member 11 arises from the U and is cantilevered from the anterior portion of the dental plate 19. It is made from rigid thermoplastic material, such as acrylic, hard durometer polypropylene, methyl vinyl acetate, ethyl vinyl acetate, polyethylene, hard durometer urethane or silicone. The overlay 17 has several holes 15 through it. The holes 15 enable the overlay 17 to be mechanically locked onto thermoplastic, thermolabile, thermoset, or elastomeric material. Such substances are acrylic, polypropylene, polyethylene, EVA, carboxylate, or any injection molded plastic, rubber or silicone

The holes 15 permit the component assembly to be attached to a dental plate 19 via an injection molded process (i.e. insert molding) or by indirect methods that require fabrication upon teeth molds in the dental laboratory, or by chairside techniques in the dental operatory whereby the orthotic is customized directly upon the patients teeth. It can be bonded, glued, heat or laser fused, or ultrasonically fused to compatible thermoplastics. The holes are an improvement over U.S. Pat. No. 5,810,013 because the holes provide mechanical retention for chemically no-adherent elastomeric and thermolabile substances.

Direct chemical adhesion to the overlay substrate (without an adhesive layer) is possible with cold cure or heat cured acrylics, glass-filled dental composites, silicone acrylic composites, and denture relining elastomers, copolymer polyesters, polyvinyl, urethane and rubber.

This shape is suitable for covering the dental arch of most adults who have a full compliment of lower teeth. The coverage is over the bicuspid, while the,molars teeth are not covered. The extension overlay has a flat smooth surface so that it can provide a flat, unindented, biting surface upon which the upper teeth may contact in a uniform manner.

The width of the mandibular overlay 17 is about 5 mm wider than the biting surface of the lower teeth so that, when it is incorporated with moldable materials to form the mandibular dental plate. This feature supports the tongue and elevates it against the palate while the jaw is being protruded.

Another important feature of the mandibular overlay is that materials that are chemically compatible with it may be bonded or adhered upon the upper contact surface. Also substances can be mechanically attached to its contact surface with the upper teeth. The ability to add thermoplastic, material to the biting surface of the overlay is an important advantage. The clinician can build onto the upper biting surface (articulating surface) ramps, ridges, contact points, guide planes, or any surface detail that will influence the motion of the lower jaw during functional and parafunctional chewing or clenching motions as long as these surface features do not interfere with the rearward adjustment of the L-shaped bracket. The act of building or not building surface characteristics into a protrusive orthotic is a matter of treatment goals. For example, incisor guidance or cuspid guidance are occlusal schemes used in orthotics to enable the disarticulation of the opposing biting surface of the teeth and the teeth on the opposite side of the dental arch.. This important property enables the biting surface of the overlay to be customized, depending on treatment objectives, so that (1) the vertical separation of the jaws can be modified, (2) the path ofjaw closure can be controlled, (3) and the excursive motion of the lower jaw can be altered to satisfy therapeutic goals.

Another important feature of the overlay is that the articulating superior surface can be lowered relative to the incisor articulating surface by grinding or by pre-fabricating it in this way in order to disclude the posterior and canine teeth during functional excursions of the mandible.

Another important feature of the overlay is a curved and raised line (i.e. incisor indicator line), which enables the operator to correctly position the overlay over the lower incisor teeth during the molding process.

The composition of the overlay enables it to be trimmed so it can be altered and adjusted to fit the contact of the upper teeth.

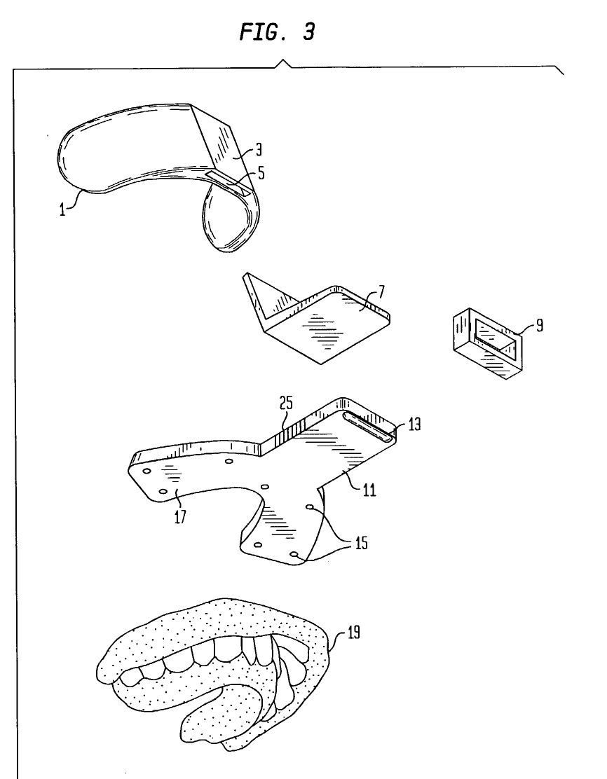

FIG. 4 shows another embodiment of the assembly shown in FIG. 3. Like numerals designate like parts in FIGS. 3 and 4. FIG. 4 does not use lip shield 1, rather an upper dental plate 2 is employed. The dental plate 2 is not affixed to the L-shaped bracket 7, rather the vertically angled portion 8 of the L-shaped bracket contacts the front of the dental plate 2. If required, rubber bands (not shown) may be connected between the upper and lower dental plates 2 and 19 to control the ability of the jaw to open. If such rubber bands are employed, hooks for the bands may be incorporated into the dental plates.

FIG. 5 shows modified overlay which can be used in either of the embodiments of FIGS. 3 or 4. In FIG. 5, the overlay 17 has its U-shaped portion formed as a dual planar superior surface, the anterior portion 10 being at a level contiguous with the extended member 11 and the posterior portion 4, 6, being at a lower level of approximately 2 mm. Said dual planar surface permits the occlusion of between two and four upper incisor teeth and enables the orthotic to disclude the cuspid, bicuspid and molar teeth on both sides of the upper dental arch during closure and/or lateral excursive motions.

### Methods of Manufacture

The preferred material is a thermolabile-thermoplastic known as Caprolactone, Polycaprolactone, 1,4-butanediolpolymer epsilon-Caprolactone, or 1,4 butanediol polyester or 2-Oxepanone. The unique property of this particular thermoplastic is that it is softened in hot water at 150 degree F. and it can then be hand molded to conform to the dental arch. When it is pressed upon the natural teeth or a mold of the teeth, it can be hand adapted and shaped. This thermoplastic is chemically adherent to acrylic resins or polycarbonate substrate and forms a chemical bond to the dental overlay of this component assembly. It can be reheated in hot water and remolded several times. Once it cools to room temperature this thermoplastic has the hardness and durability of laboratory processed dental acrylic.

### EXAMPLE 1

a direct method that requires no laboratory work consists of the following steps:

1. The doctor chooses a suitable self-curing methylmethacrylate resin, or a thermolabile-thermoplastic such as polycaprolactone that can be processed directly in the mouth or upon a mold of the mouth without the need for thermo-pressure, or specialized containment vessels. The material is prepared according to the manufacturer's directions and then it is applied to the “U” overlay while in the moldable state.

2. It is shaped it into a cylinder form which is about 1 centimeter thick; applied to the “U” overlay by completely covering the overlay and extending beyond it to cover additional teeth if necessary.

3. The moldable material is shaped to coincide with the outline of the dental arch form and an extra bulk of material is formed at the inner periphery of the overlay between the cuspid teeth so that when the patient is instructed to push against the material it will be molded to the back surface of the anterior teeth and extend inferiorly to provide a buttress against the sub-gingival alveolar bone.

Alternatively, a template or tray may be used to contain the moldable material while it is pressed upon the dentition. In this case, the bifurcated portion of the overlay may be contained within the tray or bonded to the body of the orthotic after the dental plate is made in a separate step.

4. The overlay is aligned with the midpoint of the upper lip and the lower incisors must be located directly beneath the raised indicator line.

5. The overlay is then pressed over the lower dentition and then patient is instructed to bite downward very slowly until the material covers the upper half of the teeth on the cheek aspect (4. Alternatively, if molds of the mouth are used as an intermediate step, then molds which are mounted on a hinged articulator are used lieu of the actual patient and step 5 is omitted)

6. Simultaneously, the patient is asked to push the tongue forward and from side to side in order to border mold the intraoral structure and to adapt the acrylic against the inner aspect of the mandible and on the gingival and cortical bone below the anterior teeth.

7. At this time the doctor further adapts the acrylic by molding with the fingers.

8. After the acrylic has cured, it is removed from the mouth. Cure times differ depending on the manufacturer and composition of the material.

9. The doctor then trims the orthotic with dental burs or lathes to give it,a suitable contour and less bulk.

10. Surface features may be added to the biting surface of the overlay in order to create the desired contact relationships and/or disarticulation of the bite by adding depressions or elevations onto the surface of the overlay with either methylmethacrylate resin or a compatible thermolabile thermoplastic substance.

11. The orthotic is polished to a smooth finish. The orthotic is placed back into the mouth and the functional relationship of the bite is checked in all excursive motions while the jaw is in protrusive position. The orthotic must enable the jaws to disarticulate in the posterior region during excursive motions.

12. The extraoral components are assembled onto the cantilevered extension member.

13. The holding band is loosened to allow the L bracket to slide freely upon the extension member.

14. The mandibular dental plate is placed in the mouth and the patient is instructed to bite immediately behind the raised indicator line or in an edge-to-edge posture, whichever is deemed therapeutically appropriate by the doctor.

15. The patient is instructed to move the mandible forward into protrusive posture and simultaneously the lip shield/bracket assembly is pushed against the upper subnasal tissue. If this amount of protrusion is too great then the lip shield is moved slightly forward in small increments until a comfortable jaw position is achieved.

16.The holding band is pushed against the sliding bracket in order to hold the bracket/lip shield assembly in place by the wedging action of the holding band upon the horizontal leg of the bracket..

17.If the lip shield presses too tightly into the skin, it may be re-contoured by holding it under hot water for 15 seconds, bending it with finger pressure to reduce the concavity, and then submerging it in cold water for 30 seconds.

18.The lip shield is then placed back on the vertical arm of the L bracket and positioned 2 mm below the nose. It must also be above the crowns of the upper incisor teeth..

19. Adjust the lip shield adaption to the skin surface by warming the L bracket in hot water for 15 seconds, flexing it to achieve the proper angulation and then cooling it in cold water for 30 seconds.

20. A hole is drilled through and through the middle of the lip band and cold-cured acrylic is forced into the hole and allowed to harden to lock the band to the “L” shaped bracket.

21. The position of the bracket is stabilized. This is done by adding acrylic or glue to the area just in front of the retentive band—on the under surface of the extraoral cantilever—and just behind the vertical leg of the bracket on the upper surface of the cantilever.

22. To reduce the cantilever cut off the portion anterior to the holding band. The protrusive orthotic is thus complete.

### EXAMPLE 2

a commercial laboratory method may be used with the component assembly. In this method a dental mold of the teeth is used and the overlay is luted to the biting surface of the teeth. Liquid acrylic monomer and polymer are then applied to construct the mandibular dental plate. Acrylic is thermo-pressurized or allowed to cold cure by catalytic reaction.

### EXAMPLE 3

an alternative commercial laboratory method may be used with the component assembly. In this method a dental mold of the patients teeth is used in place of the natural dentition and the construction is made by the application of preformed acrylic sheets of thermoplastic material upon the mold.

Further modifications to the methods and apparatus of the invention may be made without departing from the spirit and scope of the invention; accordingly, what is sought to be protected is set forth in the appended claims.

I claim:

1. A component assembly for a dental orthotic comprising a dental overlay having a U-shaped portion formed from a material of a first durometer, and a dental plate in part affixed to said U-shaped portion and in part self supporting, said dental plate being formed of a material having a second durometer whereby said dental orthotic provides biting surfaces of two sections with different durometers one for the biting action of the anterior teeth and another for the premolars and the molars.

2. An extraoral component assembly for a mandibular protrusive orthotic comprising a maxillary lip shield formed of a concave band of elastomeric thermoplastic material, an L-shaped mounting bracket having a horizontal leg and an obtusely angled vertical leg, and means for slidably mounting said maxillary lip shield on said obtusely angled vertical leg, said maxillary lip shield enabling the orthotic to utilize the anterior subnasal maxillary bone as a buttress to hold the mandible in protrusive posture during sleep in supine or supine lateral position.

3. The assembly of claim 2 said lip shield is shaped or molded to fit comfortably upon the soft tissue and skin covering the sub-nasal maxillary bone.

4. The assembly of claim 2 further including a horizontal extension member, said horizontal leg of said L-shaped mounting bracket being slidably mounted onto said horizontal extension member.

5. The assembly of claim 3 wherein said horizontal extension member is cantilevered several centimeters outwardly between the lips of the wearer.

6. The assembly of claim 1 wherein said dental overlay is wider than said dental plate to support the tongue as the mandible is postured in protrusive position and the patient sleeps in the supine and lateral-supine position.

7. The assembly of claim 5 further including a mandibular dental plate, a U shaped thermoplastic insert attached to said horizontal extension member, said insert being incorporated onto the biting surface of said mandibular dental plate so that the bifurcated portion of the U is rigidly bonded or mechanically locked into said mandibular dental plate.

8. The assembly of claim 4 further including a retentive band for fixing the position of said L-shaped mounting bracket on said horizontal extension member.

9. The assembly of claim 7 further including a plurality of mechanical retention holes formed in said U shaped thermoplastic insert for connecting said dental plate to said insert.

10. The assembly of claim 8 wherein the horizontal leg of said obtusely angled L bracket has a wedge shaped horizontal leg that binds to said retentive band to hold the protrusive adjustment of the lip shield.

11. In a component assembly for a dental orthotic comprising a dental overlay having a U-shaped portion formed as a dual planar superior surface the anterior portion of said surface being at a first level the posterior portion of said surface being at a lower level for permitting the occlusion of between two and four upper incisor teeth and enabling said orthotic to disclude the cuspid, bicuspid and molar teeth on both sides of the upper dental arch during closure and/or lateral excursive motions.

12. A component assembly for a mandibular protrusive orthotic comprising an unattached, intraoral maxillary dental plate for providing a resistance for mandibular protrusion, a dental overlay, an L-shaped mounting bracket having a horizontal leg attached to said dental overlay and an obtusely angled vertical leg, said vertical leg being abutted against the anterior portion of said dental plate for positioning said mandibular orthotic in forward posture.

13. The component assembly of claim 12, wherein said dental plate enables the orthotic to utilize the anterior intraoral maxillary bone and dental arch as a buttress to hold the mandible in protrusive posture without the need for interarch connections to hold the mandible in protrusive posture during sleep in supine or supine lateral position.

14. A method of joining the extraoral component to the body of a mandibular dental plate by molding thermoplastic or thermolabile material onto the surface of the U shaped overlay, which involves the following steps:
forming a heat softened thermoplastic by molding or shaped by hand manipulation upon the overlay into a defined shape;
pressing said heat softened thermoplastic upon the lower teeth in order to form a detailed impression of the teeth; and
trimming and polishing the resultant impression.

15. A method of joining the extraoral component to the body of a mandibular dental plate by applying thermoplastic or thermolabile material onto the surface of a U shaped overlay upon plaster dental molds of the patient's teeth in the dental laboratory which involves the following steps:
applying a heat softened thermoplastic or thermolabile material to the molds of the teeth by utilizing vacuum molding equipment;
pressing said heat softened thermoplastic upon the lower teeth and under vacuum pressure in order to form a detailed impression of the teeth; and
trimming and polishing the resultant impression.

16. A method of joining the extraoral component to the body of a mandibular dental plate by applying thermoplastic or thermolabile material onto the surface of a U shaped overlay upon plaster dental molds of the patient's teeth in the dental laboratory which involves the following steps:
applying chemically adherent thermoplastic resin to the molds of the teeth and upon the dental overlay of the component assembly by custom hand application in the dental laboratory;
building the resinous material upon the dental overlay in order to join it to a detailed impression of the teeth; and
trimming and polishing the resultant impression.
